(19) United States
(12) Patent Application Publication
(10) Pub. No.: US 2007/0225567 A1
(43) Pub. Date: Sep. 27, 2007

(54) ENDOSCOPE LIGHT SOURCE UNIT

(75) Inventor: Shotaro KOBAYASHI, Saitama (JP)

Correspondence Address: GREENBLUM & BERNSTEIN, P.L.C., 1950 ROLAND CLARKE PLACE, RESTON, VA, 20191, US

(73) Assignee: PENTAX Corporation, Tokyo (JP)

(21) Appl. No.: 11/688,978

(22) Filed: Mar. 21, 2007

(30) Foreign Application Priority Data
Mar. 24, 2006 (JP) 2006-082796

(51) Int. Cl. A61B 1/06

(52) U.S. Cl. 600/178; 600/181; 600/182

(57) ABSTRACT

An endoscope light source unit including a light source lamp and an infrared-cut filter for cutting out light rays with wavelengths of a near-infrared region which are included in illumination light emitted from the light source lamp, the endoscope light source unit includes a transparent conductive coating formed on at least one of front and rear surfaces of the infrared-cut filter; at least one pair of electrode portions provided on the one of the front and rear surfaces of the infrared-cut filter in a vicinity of a rim thereof on opposite sides of the transparent conductive coating, respectively, to be electrically connected to the transparent conductive coating; and an electrical-characteristic detector which measures an electrical characteristic of the pair of electrode portions to detect whether the electrical characteristic exceeds a predetermined threshold value.

### BACKGROUND OF THE INVENTION

1. Field of the Invention

The present invention relates to an endoscope light source unit.

2. Description of the Related Art

Conventional endoscope light source units are usually provided between a light source lamp and the incident end face of a scope light guide (light guide for lighting) with an infrared-cut filter for cutting out light rays with wavelengths of the near-infrared region which are included in the illumination light emitted from the light source lamp. This configuration is disclosed in, e.g., Japanese patent No. 3,068,702.

A glass substrate onto which a dielectric material is evaporated in layers is generally used as an infrared-cut filter. However, if the infrared-cut filter repeatedly receives heat radiation from the light source lamp for a long period of time, the dielectric multilayer and the glass substrate itself may be damaged by thermal deterioration. If the endoscope light source unit continues to be used with such an infrared-cut filter thus damaged, the temperature of the illumination light incident on the scope light guide rises excessively, which may damage the endoscope and burn a patient.

### SUMMARY OF THE INVENTION

The present invention provides an endoscope light source unit which can prevent the temperature of the illumination light incident upon the scope light guide from rising excessively by immediately detecting a breakage of the infrared-cut filter without delay upon.

According to an aspect of the present invention, an endoscope light source unit is provided, including a light source lamp and an infrared-cut filter positioned between the light source lamp and an incident end face of a light guide for lighting of an endoscope connected to the endoscope light source unit, the infrared-cut filter cutting out light rays with wavelengths of a near-infrared region which are included in illumination light emitted from the light source lamp, wherein the endoscope light source unit includes a transparent conductive coating formed on at least one of front and rear surfaces of the infrared-cut filter; at least one pair of electrode portions provided on the one of the front and rear surfaces of the infrared-cut filter in a vicinity of a rim thereof on opposite sides of the transparent conductive coating, respectively, to be electrically connected to the transparent conductive coating; and an electrical-characteristic detector which measures an electrical characteristic of the pair of electrode portions to detect whether the electrical characteristic exceeds a predetermined threshold value.

It is desirable for the electrical characteristic between the pair of electrode portions includes one of an increase in electrical resistance, a decrease in electrical current and an increase in electrical voltage.

It is desirable for the endoscope light source unit to include an indicator which indicates that the electrical characteristic exceeds the predetermined threshold value upon the electrical-characteristic detector detecting that the electrical characteristic exceeds the predetermined threshold value.

It is desirable for the indicator to include one of a monitor and a warning light which visually indicates a warning that the electrical characteristic exceeds the predetermined threshold value.

It is desirable for the indicator to auditorily indicate a warning that the electrical characteristic exceeds the predetermined threshold value.

It is desirable for the endoscope light source unit to include a controller which operates to reduce an amount of emission of light rays with the wavelengths of the near-infrared region which travel toward the incident end face of the light guide upon the electrical-characteristic detector detecting that the electrical characteristic exceeds the predetermined threshold value.

It is desirable for the controller to control the operation of the light source lamp to reduce the amount of emission of light rays.

It is desirable for the endoscope light source unit to include an adjustable diaphragm, positioned between the light source lamp and the infrared-cut filter, for limiting the amount of emission of the light rays with the wavelengths of the near-infrared region that travel toward the incident end face of the light guide, wherein the controller controls an aperture size of the adjustable diaphragm.

It is desirable for the endoscope light source unit to include an auxiliary lamp, provided in addition to the light source lamp, which emits illumination light including relatively small amount of light rays with wavelengths of a near-infrared region, wherein the controller operates to make the illumination light which is emitted from the auxiliary lamp incident upon the incident end face of the light guide instead of the illumination light which is emitted from the light source lamp.

It is desirable for the transparent conductive coating to be formed on each of the front and rear surfaces of the infrared-cut filter, wherein the pair of electrode portions is provided on each of the front and rear surfaces of the infrared-cut filter, and the pair of electrode portions on the front surface of the infrared-cut filter and the pair of electrode portions on the rear surface of the infrared-cut filter are orientated in a different directions.

It is desirable for the different directions to be substantially orthogonal to each other.

It is desirable for the transparent conductive coating on the infrared-cut filter to be formed thereon in a band shape.

It is desirable for the transparent conductive coating on the front surface of the infrared-cut filter to be formed thereon in a band shape elongated in a first direction, and for the transparent conductive coating on the rear surface of the infrared-cut filter to be formed thereon in a band shape elongated in a second direction substantially orthogonal to the first direction.

It is desirable for the transparent conductive coating to be formed on only one of the front and rear surfaces of the infrared-cut filter. The pair of electrode portions includes two pairs of electrode portions which are formed on the only one of the front and rear surfaces of the infrared-cut filter in different directions orthogonal to each other, respectively.

It is desirable for the endoscope light source unit to include a filter holding frame for holding a rim of the infrared-cut filter to fix the infrared-cut filter to a stationary portion of the endoscope light source unit, the filter holding frame having a pair of conductive frame member, wherein the conductive frame members of the filter holding frame are in pressing contact with the pair of electrode portions, respectively.

It is desirable for at least one pair of filter holding frames for holding a rim of the infrared-cut filter to fix the infrared-cut filter to a stationary portion of the endoscope light source unit, each of the pair of filter holding frames having a conductive frame member, wherein the conductive frame members of the pair of filter holding frames are in pressing contact with the pair of electrode portions, respectively.

It is desirable for a lead wire which is connected to the conductive frame member of each of the pair of filter holding frames to extend outwardly therefrom.

It is desirable for the at least one pair of electrode portions to include two pairs of electrode portions, wherein the at least one pair of filter holding frames include two pairs of filter holding frames which are arranged at substantially equi-angular intervals, and the conductive frame members of the two pairs of filter holding frames are in pressing contact with the two pair of electrode portions, respectively.

In an embodiment, an endoscope light source unit is provided, including a light source; a condenser optical system which converges an incident light thereon, which is emitted from the light source, to a point substantially at an incident end face of a light guide of an endoscope connected to the endoscope light source unit; an infrared-cut filter positioned between the light source and the condenser lens; a transparent conductive coating formed on at least one of front and rear surfaces of the infrared-cut filter; at least two pairs of electrode portions provided on the infrared-cut filter at a rim thereof around the transparent conductive coating to be electrically connected to the transparent conductive coating; and a electrical-characteristic detector which measures electrical characteristic between each pair of the two pairs of electrode portions to detect whether the electrical characteristic exceeds a predetermined threshold value.

It is desirable for the transparent conductive coating to be formed on each of the front and rear surfaces of the infrared-cut filter, and for the two pairs of electrode portions are formed on the front and rear surfaces of the infrared-cut filter to be electrically connected to the transparent conductive coatings on the front and rear surfaces of the infrared-cut filter, respectively.

According to the present invention, the temperature of the illumination light incident upon the scope light guide can be prevented from rising excessively by immediately detecting a breakage of the infrared-cut filter without delay upon because the electric resistance between a pair of electrode portions on the infrared-cut filter which are arranged on opposite sides of the transparent conductive coating on the infrared-cut filter increases upon the infrared-cut filter being damaged and this increase in electric resistance is detected by the electrical-characteristic detector.

The present disclosure relates to subject matter contained in Japanese Patent Application No. 2006-82796 (filed on Mar. 24, 2006), which is expressly incorporated herein in its entirety.

### BRIEF DESCRIPTION OF THE DRAWINGS

The present invention will be discussed below in detail with reference to the accompanying drawings, in which:

FIG. 1 is a cross sectional view of an internal portion of a first embodiment of an endoscope light source unit according to the present invention, taken along a plane orthogonal to the optical axis of an infrared-cut filter (taken along I-I line shown in FIG. 2);

FIG. 2 is a schematic diagram of the first embodiment of the endoscope light source unit to which an endoscope is attached;

FIG. 3 is a front view of the infrared-cut filter provided in the first embodiment of the endoscope light source unit;

FIG. 4 is a rear view of the infrared-cut filter shown in FIG. 3;

FIG. 5 is a perspective view of a filter holding frame of the first embodiment of the endoscope light source unit;

FIG. 6 is a flow chart showing operations of a program performed in a controller provided in the first embodiment of the endoscope light source unit;

FIG. 7 is a schematic diagram of a second embodiment of the endoscope light source unit to which an endoscope is attached;

FIG. 8 is a view similar to that of FIG. 7, showing an operational status of the second embodiment of the endoscope light source unit in the case where the infrared-cut filter is damaged; and

FIG. 9 is a front view of the infrared-cut filter provided in a third embodiment of the endoscope light source unit.

### DESCRIPTION OF THE PREFERRED EMBODIMENTS

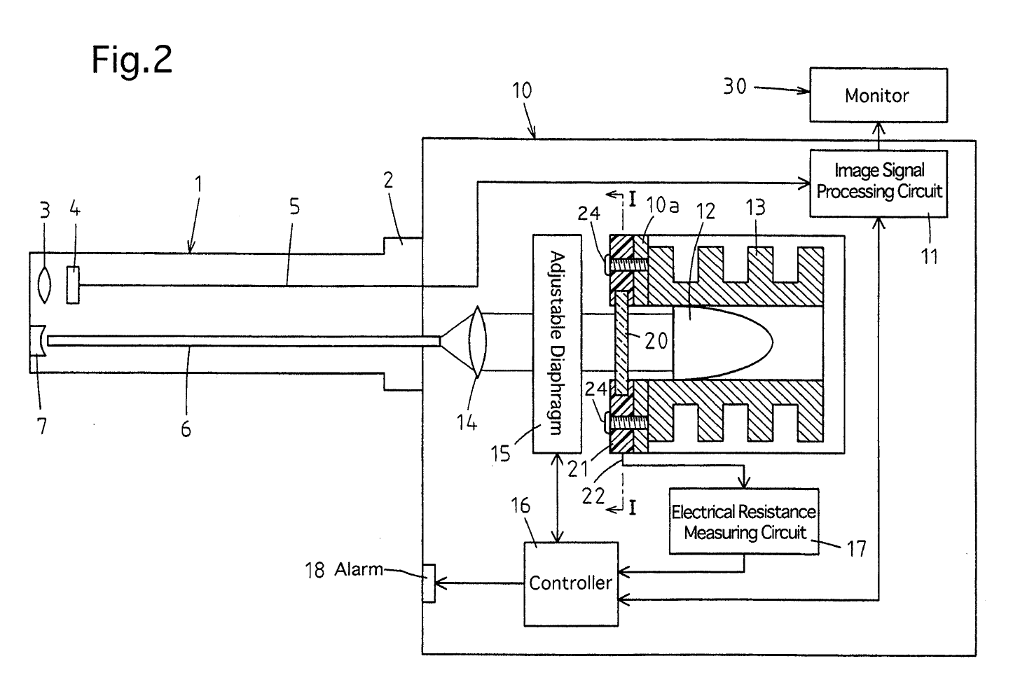

FIG. 2 shows a state where a connector 2 of an endoscope 1 is connected to a first embodiment of an endoscope light source unit 10 according to the present invention. The light source unit 10 also serves as a video processor. The connector 2 can be freely attached to and detached from the light source unit 10.

The endoscope 1 is provided in the distal end of the insertion portion thereof with an objective optical system 3 and a solid-state image pickup device 4 positioned in a plane onto which an object image is projected via the objective optical system 3. The endoscope 1 is provided therein with a signal cable 5 for transmitting image signals output from the solid-state image pickup device 4. The signal cable 5 is connected to a video signal processing circuit 11 in the light source unit 10 via the connector 2 so that endoscopic video images are displayed on a monitor 30 in which the video signal is input from the video signal processing circuit 11.

The endoscope 1 is provided therein with a light-guide fiber bundle 6 for lighting which is inserted into the endoscope 1 to pass therethrough. The exit end of the light-guide fiber bundle 6 is positioned immediately behind a light distribution lens 7 provided in the distal end of the insertion portion of the endoscope 1 so that an object to be viewed on the monitor 30 is illuminated by illumination light emitted from the exit end of the light-guide fiber bundle 6. The incident end of the light-guide fiber bundle 6 is inserted into the light guide unit 10 when the endoscope 1 is connected to the light guide unit 10 via the connector 2.

The light source unit 10 is provided therein with a light source lamp 12 (e.g., xenon lamp) which emits illumination light that is supplied to the light-guide fiber bundle 6. The light source lamp 12 is surrounded by a heat sink 13 for dissipating heat produced by the light source lamp 12.

The light source unit 10 is provided between the light source lamp 12 and the incident end face of the light-guide fiber bundle 6 with a condenser lens 14 which is used to converge the incident light, which is emitted from the light source lamp 12, to a point in the vicinity of the incident end face of the light-guide fiber bundle 6.

The light source unit 10 is provided between the condenser lens 14 and the light source lamp 12 with an adjustable diaphragm 15. The cross-sectional area of a bundle of illumination light which is emitted from the light source lamp 12 toward the condenser lens 14 can be freely changed by adjusting the aperture of the adjustable diaphragm 15 to partly intercept the bundle of illumination light passing through the adjustable diaphragm 15. The operation of the adjustable diaphragm 15 is controlled with control signals output from a controller 16 including an MPU (micro processing unit).

The light source unit 10 is provided between the light source lamp 12 and the adjustable diaphragm 15 with a disk-shaped infrared-cut filter 20 adopted for cutting out light rays with wavelengths of the near-infrared region which are included in the illumination light emitted from the light source lamp 12. The infrared-cut filter 20 is a conventional infrared-cut filter which partly reflects light rays with wavelengths of the near-infrared region while absorbing most of the remaining near-infrared light rays.

The infrared-cut filter 20 is fixed to the heat sink 13 via four filter holding frames 21, a support frame 10a and a plurality of set screws 24 (only two of which are shown in FIG. 2). More specifically, the support frame 10a is fixed to the front end of the heat sink 13 by conventional fixing means such as set screws or welding, and the infrared-cut filter 20 is secured to the support frame 10a by the plurality of set screws 24 with the outer edge of the infrared-cut filter 20 being held by the four filter holding frames 21. An insulated lead wire 22 extends outwardly from each filter holding frame 21 to be connected to an electrical resistance measuring circuit (electrical-characteristic detector) 17 provided in the light source unit 10. It is possible that both the four filter holding frames 21 and the support frame 10a be secured to the front end of the heat sink 13 by the plurality of set screws 24.

A detection signal output from the electrical resistance measuring circuit 17 is input to the controller 16. Additionally, other signals are sent to and received from the video signal processing circuit 11, and the controller 16 outputs a control signal to an alarm 18 provided on a front panel, or the like, of the light source unit 10.

The infrared-cut filter 20, which is installed in the light source unit 10 in the above described manner, is provided on the front and rear sides thereof, with transparent and colorless front and rear conductive coating 23A and 23B, respectively, made from, e.g., ITO (indium tin oxide).

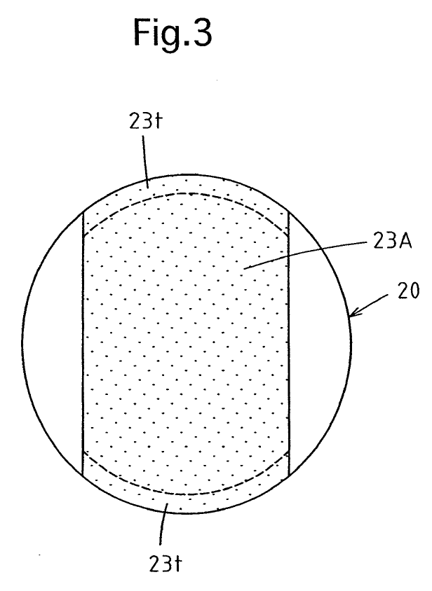

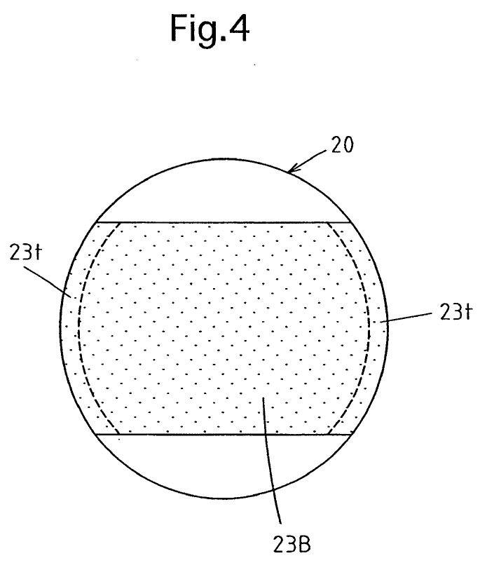

FIGS. 3 and 4 show the front and rear surfaces of the infrared-cut filter 20, respectively. As shown in FIG. 3, the front conductive coating 23A is formed on the front surface of the infrared-cut filter 20 in a vertically-elongated band shape. As shown in FIG. 4, the rear conductive coating 23B is formed on the rear surface of the infrared-cut filter 20 in a horizontally-elongated band shape that is elongated in a direction orthogonal to the direction of elongation of the front conductive coating 23A.

The infrared-cut filter 20 is provided, on the front surface thereof on opposite sides of the front conductive coating 23A along the rim of the infrared-cut filter 20, with a pair of electrode portions (front pair of electrode portions) 23t, respectively, which are electrically connected to the front conductive coating 23A. Likewise, the infrared-cut filter 20 is provided, on the rear surface thereof on opposite sides of the rear conductive coating 23B along the rim of the infrared-cut filter 20, with another pair of electrode portions (rear pair of electrode portions) 23t, respectively, which are electrically connected to the rear conductive coating 23B. The pair of electrode portions 23 on the front surface of the infrared-cut filter 20 are orientated in a different direction to, i.e., orthogonal to, that of the pair of electrode portions 23t on the rear surface of the infrared-cut filter 20.

In other words, the infrared-cut filter 20 is provided, on opposite sides of the front conductive coating 23A at upper and lower ends thereof, with the front pair of electrode portions 23t, respectively. Likewise, the infrared-cut filter 20 is provided, on opposite sides of the rear conductive coating 23B at right and left ends thereof, with the rear pair of electrode portions 23t, respectively.

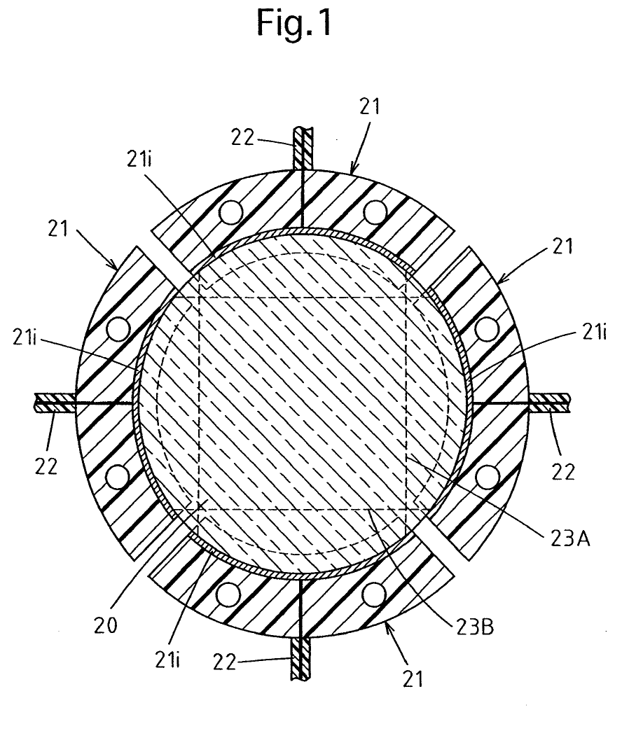

FIG. 1 is a cross sectional view of an internal portion of the light source unit 10, taken along a plane through the infrared-cut filter 20 orthogonal to the optical axis (taken along I-I line shown in FIG. 2). The infrared-cut filter 20 is fixed to the heat sink 13 inside the light source unit 10 with the rim of the infrared-cut filter 20 being held by the four filter holding frames 21 spaced at regular intervals in a circumferential direction of the infrared-cut filter 20.

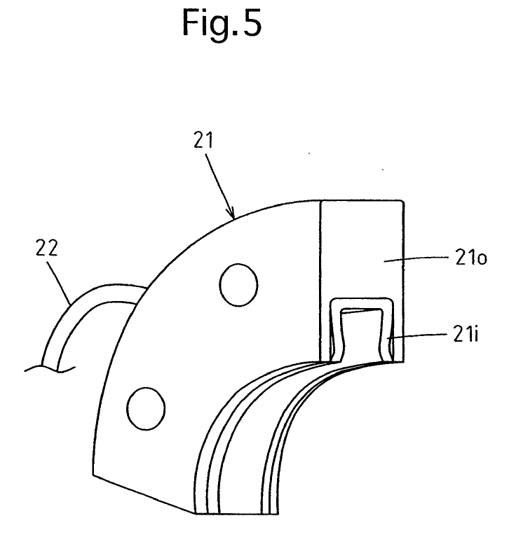

As shown in also FIG. 5 that shows one filter holding frames 21, each filter holding frame 21 is in the shape of a circular arc as viewed from either of the front and the rear thereof, and is provided with an insulating member 21o and a conductive frame member 21i. More specifically, the insulating member 21o is provided along a radially inner end surface thereof with an arc-shaped recess which is recessed radially outwards, and the conductive frame member 21i having a substantially U-shape in cross section is fixedly positioned inside of the arc-shaped recess of the insulating member 21o. The conductive frame member 21i is made of a springy conductive metal capable of holding the rim of the infrared-cut filter 20 while being in pressing contact with the associated electrode portion 23t, and is formed in a circular arc shape, the central angle of which is in the range of approximately 80 to 85 degrees. The insulated lead wire 22 of each filter holding frame 21, which is connected to the conductive frame member 21i thereof, extends outwardly from the radially outer edge of the associated insulating member 21o.

Consequently, as shown in FIG. 1, among the four insulated lead wires 22, the two insulated lead wires 22 which extend from the two filter holding frames 21 positioned on the upper and lower ends of the infrared-cut filter 20 are connected to the front pair of electrode portions 23t, which are positioned at the vertically opposite ends of the front conductive coating 23A, respectively, while being electrically insulated from the rear conductive coating 23B, and the remaining two insulated lead wires 22 that extend from the two filter holding frames 21 positioned on the right and left ends of the infrared-cut filter 20 are connected to the rear pair of electrode portions 23t, which are positioned at the horizontally opposite ends of the rear conductive coating 23B, respectively, while being electrically insulated from the front conductive coating 23A.

As a result, electrical resistance (resistance value) R1 between the upper and lower ends of the front conductive coating 23A on the front surface of the infrared-cut filter 20 is calculated in the electrical resistance measuring circuit 17 via the two lead wires 22 which are connected to the front pair of electrode portions 23t that are positioned at the vertically opposite ends of the front conductive coating 23A, respectively, so that an increase (upsurge) of the electrical resistance R1 can be detected without delay in the event of a crack, a fracture or the like extending in the horizontal direction (including near-horizontal directions inclined to the horizontal direction) occurring in the infrared-cut filter 20.

Additionally, electrical resistance (resistance value) R2 between the right and left ends of the rear conductive coating 23B on the rear surface of the infrared-cut filter 20 is calculated in the electrical resistance measuring circuit 17 via the two lead wires 22 which are connected to the rear pair of electrode portions 23t that are positioned at the horizontally opposite ends of the rear conductive coating 23B, respectively, so that an increase (upsurge) of the electrical resistance R2 can be detected without delay in the event that a crack, a fracture or the like extending in the vertical direction (including near-vertical directions inclined to the vertical direction) occurs in the infrared-cut filter 20.

The sum of the electrical resistance R1 between the upper and lower ends of the front conductive coating 23A on the front surface of the infrared-cut filter 20 and the electrical resistance R2 between the right and left ends of the rear conductive coating 23B on the rear surface of the infrared-cut filter 20 (=R1+R2) suddenly increases upon an occurrence of a crack, fracture or the like extending in either of the horizontal and vertical directions, and accordingly, there is a big difference between the sum of the electrical resistances R1 and R2 when no crack, fracture or the like occurs in either surface of the infrared-cut filter 20 and the sum of the electrical resistances R1 and R2 when a crack, a fracture or the like occurs in either surface of the infrared-cut filter 20.

Accordingly, an intermediate value (not necessarily the precise central value) between the sum of the electrical resistances R1 and R2 when no crack, fracture or the like occurs in either surface of the infrared-cut filter 20 and the sum of the electrical resistances R1 and R2 when a crack, a fracture or the like occurs in either surface of the infrared-cut filter 20 is set and stored in advance in the controller 16 as a threshold value R0. Upon the occurrence of a crack or fracture in the infrared-cut filter 20, since the electrical characteristics between the front and rear conductive coatings 23A and 23B change, such a crack or fracture can be detected by detecting these electrical characteristics. Alternatively, instead of detecting electrical resistance, a drop (decrease) in electrical current can be alternatively detected.

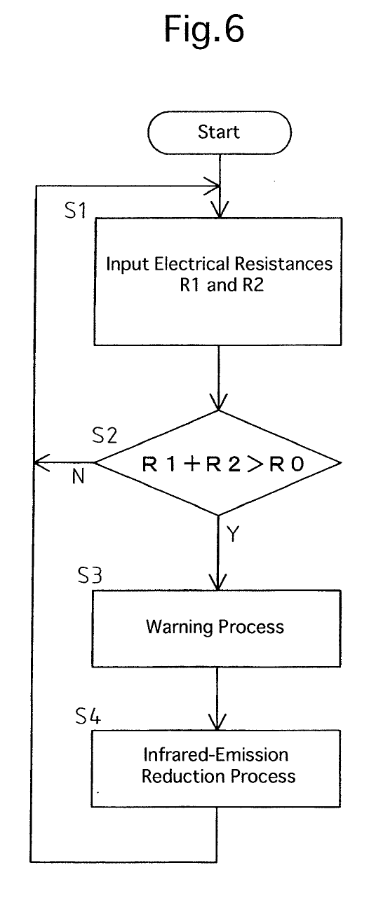

FIG. 6 is a flow chart showing operations of a process performed in the controller 16. This process is started upon the light source lamp 12 being turned ON.

Upon control entering this process, the electrical resistance R1 between the upper and lower ends of the front conductive coating 23A on the front surface of the infrared cut filter 20 and the electrical resistance R2 between the right and left ends of the rear conductive coating 23B on the rear surface of the infrared cut filter 20 are input at step S1.

Subsequently, the sum (R1+R2) is compared with the threshold value R0 to determine whether the threshold value R0 is smaller than the sum (R1+R2) at step S2. If the sum (R1+R2) is equal to or smaller than the threshold value R0 (if NO at step S2), control returns to step S1 to repeat the operations at steps S1 and S2. If the sum (R1+R2) is greater than the threshold value R0 (if YES at step S2), control proceeds to step S3 at which a warning process is performed, and subsequently, an infrared-emission reduction process, in which the amount of emission of light rays with wavelengths of the near-infrared region which travel toward the incident end face of the light-guide fiber bundle 6 is reduced, is performed at step S4. Thereafter, control returns to step S1 to repeat the operations at steps S1 through S4.

Specifically, the warning process performed at step S3 can be, e.g., a process of visually indicating a warning light provided in the alarm 18 or a warning sign displayed on the monitor 30 or the like, or a process of auditorily indicating a warning (e.g., a process of generating an audible alert by a beeper or the like provided in the alarm 18). Alternatively, the warning process performed at step S3 can include both such visual and auditory processes.

Additionally, the infrared-emission reduction process performed at step S4 can be, e.g., a process of reducing the amount of light emission of the light source lamp 12 by controlling the output of a power supply (not shown) for the light source lamp 12, or a process of controlling the aperture of the adjustable diaphragm 15 to reduce the aperture size thereof.

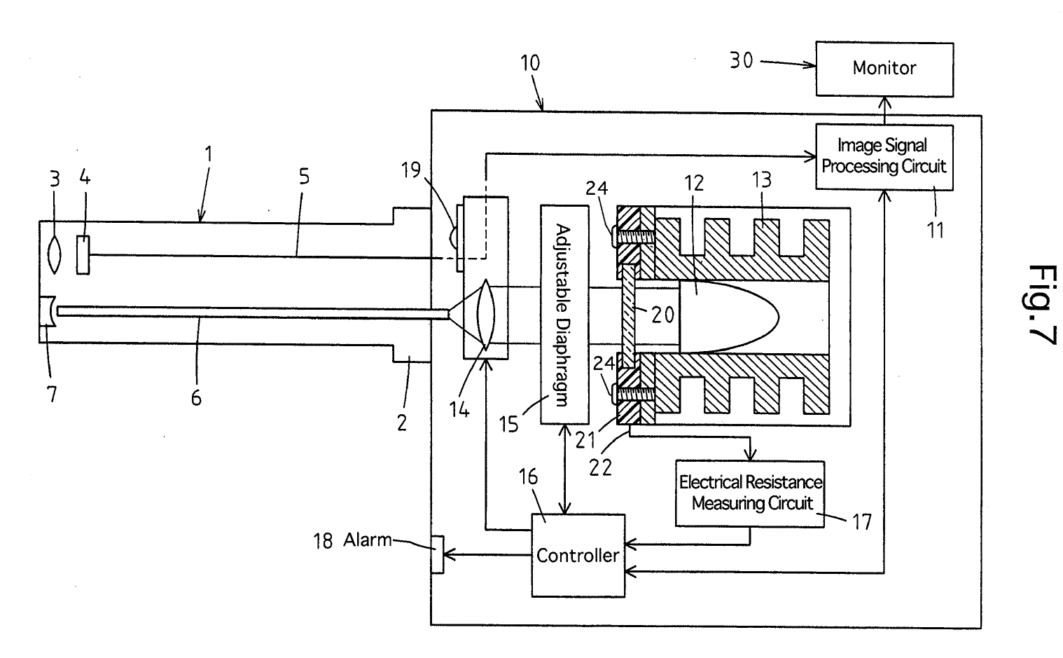

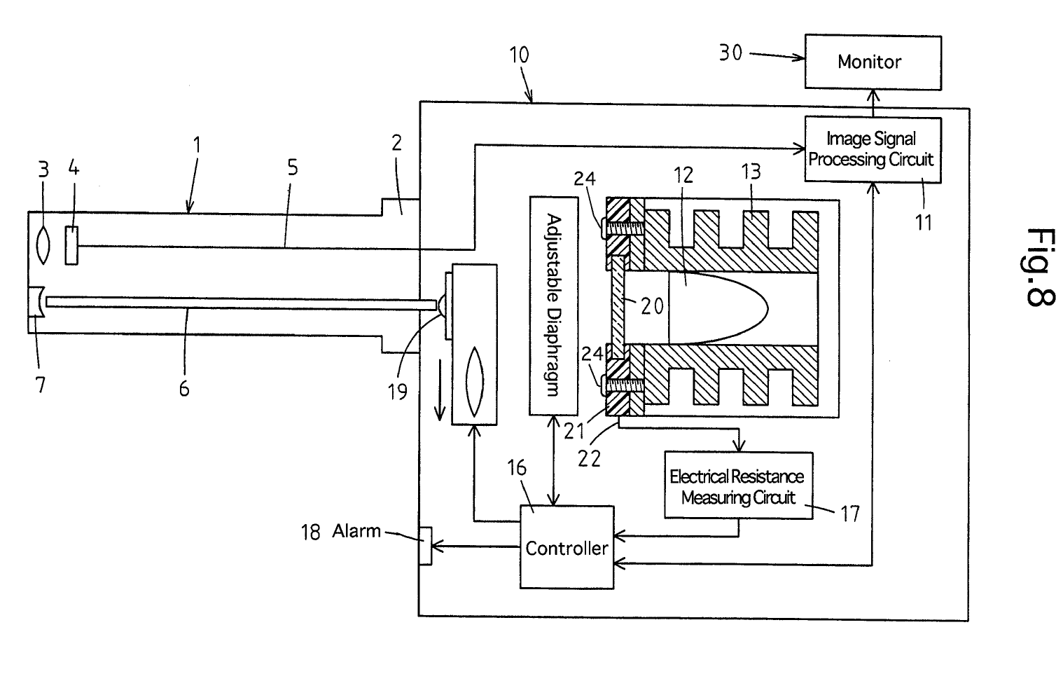

In a second embodiment of the endoscope light source unit shown in FIG. 7, the light source unit 10 is further provided therein, in addition to the light source lamp 12, with a movable auxiliary light source 19 which is movable with the condenser lens 14 and emits illumination light including relatively small amount of light rays with wavelengths of the near-infrared region such as light emission of an LED. In this embodiment, in the infrared-emission reduction process performed at step S4, it is possible to turn OFF the light source lamp 12 (or fully shut the adjustable diaphragm 15) and simultaneously move the movable auxiliary light source 19 to a position where the movable auxiliary light source 19 faces the incident surface of the light-guide fiber bundle 6 by a moving mechanism (not shown) so that the light emitted from the movable auxiliary light source 19 is incident on the incident surface of the light-guide fiber bundle 6 as shown in FIG. 8.

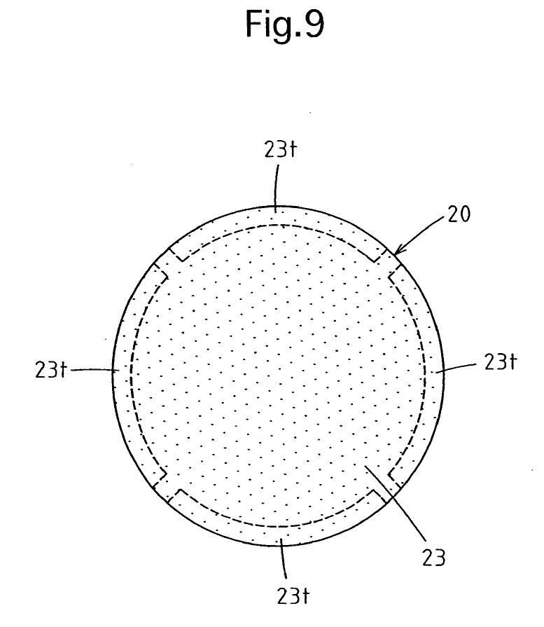

The present invention is not limited solely to the particular embodiments described above. For instance, as shown in FIG. 9, the infrared cut filter 20 can be provided over only one of the front and rear surfaces thereof with a conductive coating 23 and further provided, along the outer edge of the infrared cut filter 20 at substantially equi-angular intervals, with four electrode portions 23t, respectively: a pair of electrode portions (right and left electrode portions) 23t and another pair of electrode portions (upper and lower electrode portions) 23t which are positioned along two directions substantially orthogonal to each other, respectively. In this embodiment, the electrical resistance between the upper and lower ends of the conductive coating 23 (between the upper and lower electrode portions 23t) and the electrical resistance between the right and left ends of the conductive coating 23 (between the right and left electrode portions 23t) can be measured to obtain an effect similar to that obtained in each of the above described embodiments.

Upon the occurrence of a crack or fracture in the infrared cut filter 20 so that a crack or fracture occurs in the transparent conductive coating 23A, 23B or 23, since the electrical characteristics between the front and rear conductive coatings 23A and 23B (or within conductive coating 23) change, such a crack or fracture can be detected by detecting these electrical characteristics.

Although in the above illustrated embodiments an increase in electrical resistance was detected in order to determine cracks or fractures in the infrared-cut filter 20, it is possible to detect cracks or fractures in the infrared-cut filter 20 via detecting a decrease in electrical current or an increase in voltage (difference of electric potential).

Obvious changes may be made in the specific embodiments of the present invention described herein, such modifications being within the spirit and scope of the invention claimed. It is indicated that all matter contained herein is illustrative and does not limit the scope of the present invention.

What is claimed is:

1. An endoscope light source unit including a light source lamp and an infrared-cut filter positioned between said light source lamp and an incident end face of a light guide for lighting of an endoscope connected to said endoscope light source unit, said infrared-cut filter cutting out light rays with wavelengths of a near-infrared region which are included in illumination light emitted from said light source lamp, wherein said endoscope light source unit comprises:
a transparent conductive coating formed on at least one of front and rear surfaces of said infrared-cut filter;
at least one pair of electrode portions provided on said one of said front and rear surfaces of said infrared-cut filter in a vicinity of a rim thereof on opposite sides of said transparent conductive coating, respectively, to be electrically connected to said transparent conductive coating; and
an electrical-characteristic detector which measures an electrical characteristic of said pair of electrode portions to detect whether said electrical characteristic exceeds a predetermined threshold value.

2. The endoscope light source unit according to claim 1, wherein said electrical characteristic between said pair of electrode portions comprises one of an increase in electrical resistance, a decrease in electrical current and an increase in electrical voltage.

3. The endoscope light source unit according to claim 1, further comprising an indicator which indicates that said electrical characteristic exceeds said predetermined threshold value upon said electrical-characteristic detector detecting that said electrical characteristic exceeds said predetermined threshold value.

4. The endoscope light source unit according to claim 3, wherein said indicator comprises one of a monitor and a warning light which visually indicates a warning that said electrical characteristic exceeds said predetermined threshold value.

5. The endoscope light source unit according to claim 3, wherein said indicator auditorily indicates a warning that said electrical characteristic exceeds said predetermined threshold value.

6. The endoscope light source unit according to claim 1, further comprising a controller which operates to reduce an amount of emission of light rays with said wavelengths of the near-infrared region which travel toward said incident end face of said light guide upon said electrical-characteristic detector detecting that said electrical characteristic exceeds said predetermined threshold value.

7. The endoscope light source unit according to claim 6, wherein said controller controls the operation of said light source lamp to reduce said amount of emission of light rays.

8. The endoscope light source unit according to claim 6, further comprising an adjustable diaphragm, positioned between said light source lamp and said infrared-cut filter, for limiting said amount of emission of said light rays with said wavelengths of the near-infrared region that travel toward said incident end face of said light guide,
wherein said controller controls an aperture size of said adjustable diaphragm.

9. The endoscope light source unit according to claim 6, further comprising an auxiliary lamp, provided in addition to said light source lamp, which emits illumination light including relatively small amount of light rays with wavelengths of a near-infrared region,
wherein said controller operates to make said illumination light which is emitted from said auxiliary lamp incident upon said incident end face of said light guide instead of said illumination light which is emitted from said light source lamp.

10. The endoscope light source unit according to claim 1, wherein said transparent conductive coating is formed on each of said front and rear surfaces of said infrared-cut filter,
wherein said pair of electrode portions is provided on each of said front and rear surfaces of said infrared-cut filter, and
wherein said pair of electrode portions on said front surface of said infrared-cut filter and said pair of electrode portions on said rear surface of said infrared-cut filter are orientated in a different directions.

11. The endoscope light source unit according to claim 10, wherein said different directions are substantially orthogonal to each other.

12. The endoscope light source unit according to claim 11, wherein said transparent conductive coating on said infrared-cut filter are formed thereon in a band shape.

13. The endoscope light source unit according to claim 11, wherein said transparent conductive coating on said front surface of said infrared-cut filter is formed thereon in a band shape elongated in a first direction, and
wherein said transparent conductive coating on said rear surface of said infrared-cut filter is formed thereon in a band shape elongated in a second direction substantially orthogonal to said first direction.

14. The endoscope light source unit according to claim 1, wherein said transparent conductive coating is formed on only one of said front and rear surfaces of said infrared-cut filter,
wherein said pair of electrode portions comprises two pairs of electrode portions which are formed on said only one of said front and rear surfaces of said infrared-cut filter in different directions orthogonal to each other, respectively.

15. The endoscope light source unit according to claim 1, further comprising a filter holding frame for holding a rim of said infrared-cut filter to fix said infrared-cut filter to a stationary portion of said endoscope light source unit, said filter holding frame having a pair of conductive frame member,
wherein said conductive frame members of said filter holding frame are in pressing contact with said pair of electrode portions, respectively.

16. The endoscope light source unit according to claim 1, further comprising at least one pair of filter holding frames for holding a rim of said infrared-cut filter to fix said infrared-cut filter to a stationary portion of said endoscope light source unit, each of said pair of filter holding frames having a conductive frame member,
wherein said conductive frame members of said pair of filter holding frames are in pressing contact with said pair of electrode portions, respectively.

17. The endoscope light source unit according to claim 16, wherein a lead wire which is connected to said conductive frame member of each of said pair of filter holding frames extends outwardly therefrom.

18. The endoscope light source unit according to claim 16, wherein said at least one pair of electrode portions comprise two pairs of electrode portions,
wherein said at least one pair of filter holding frames comprise two pairs of filter holding frames which are arranged at substantially equi-angular intervals, and
wherein said conductive frame members of said two pairs of filter holding frames are in pressing contact with said two pair of electrode portions, respectively.

19. An endoscope light source unit comprising:
a light source;
a condenser optical system which converges an incident light thereon, which is emitted from said light source, to a point substantially at an incident end face of a light guide of an endoscope connected to said endoscope light source unit;
an infrared-cut filter positioned between said light source and said condenser lens;
a transparent conductive coating formed on at least one of front and rear surfaces of said infrared-cut filter;
at least two pairs of electrode portions provided on said infrared-cut filter at a rim thereof around said transparent conductive coating to be electrically connected to said transparent conductive coating; and
a electrical-characteristic detector which measures electrical characteristic between each pair of said two pairs of electrode portions to detect whether said electrical characteristic exceeds a predetermined threshold value.

20. The endoscope light source unit according to claim 19, wherein said transparent conductive coating is formed on each of said front and rear surfaces of said infrared-cut filter, and
wherein said two pairs of electrode portions are formed on said front and rear surfaces of said infrared-cut filter to be electrically connected to said transparent conductive coatings on said front and rear surfaces of said infrared-cut filter, respectively.
